(19) United States
(12) Patent Application Publication
(10) Pub. No.: US 2014/0276306 A1
(43) Pub. Date: Sep. 18, 2014

(54) PHYSICAL THERAPY SUPPORT DEVICE AND HARNESS

(71) Applicant: RETRAINER INC., Las Vegas, NV (US)

(72) Inventor: Edward Ronald Dreske, Taylorsville, UT (US)

(73) Assignee: RETRAINER INC., Las Vegas, NV (US)

(21) Appl. No.: 13/836,815

(22) Filed: Mar. 15, 2013

(51) Int. Cl. A61H 3/00; A61F 5/02

(52) U.S. Cl. CPC A61H 3/008; A61F 5/02
USPC 602/19

(57) ABSTRACT

A harness assembly for suspending a user. The assembly includes a support. The support includes a back plate having a shape corresponding to a user's back for supporting the user's back in a predetermined configuration. A torso connector is fastened to the support for engaging the user's torso and releasably fastening the support to the user's torso adjacent the user's back. A leg loop is connected to the support. The leg loop is sized for surrounding the user's leg to fasten the support to the user's leg. The assembly includes a suspender that is extendable outward from the support for suspending the user fastened to the support.

### BACKGROUND

The present invention generally relates to physical therapy support devices, and more particularly to a physical therapy support devices having a mobile framework and harness assembly.

Physical therapy is useful to rehabilitate patients and allow them to recover capability after injury and/or surgery. When a patient is unable to walk or stand upright, attendants are needed to support the patient during therapy or between therapy stations. These attendants may be diverted from other duties, potentially reducing patient care. Even if the attendants are not diverted from other duties, they must be paid, increasing the cost of therapy. Thus, there is a need for a device capable of supporting patients during therapies and between therapy stations to eliminate or reduce attendants needed during physical therapy.

### SUMMARY

In one aspect, the present invention includes a harness assembly comprising a support. The support includes a back plate having a shape corresponding to a user's back for supporting the user's back in a predetermined configuration. A torso connector is connected to the support for engaging the user's torso and releasably fastening the support to the user's torso adjacent the user's back. A leg loop is connected to the support. The leg loop is sized for surrounding the user's leg to fasten the support to the user's leg. A suspender is extendable outward from the support for suspending the user fastened to the support.

In another aspect of the invention, a harness assembly for supporting a back of a user comprises a support. The support includes a back plate having a shape corresponding to a user's back for supporting the user's back in a predetermined configuration. The back plate has a vertical length extending in use along a user's spine from a lower portion adjacent a user's waist upward to an upper position adjacent a user's scapula. The assembly includes a torso connector connected to the support for engaging the user's torso and releasably fastening the support to the user's torso adjacent the user's back, and a shoulder strap connected to the torso connector and looped above a shoulder of the user.

In still another aspect of the present invention, a physical therapy support device comprises a framework sized for at least partially surrounding a user. The framework has a frame member and a plurality of legs extending downward from the frame member to casters positioned at lower ends of the legs. A hoist is secured to the frame member for adjustably suspending the user within the framework. The hoist comprises a suspension connector releasably connectable to the user and a suspension cable connected to the connector and adapted to selectively raise and lower the suspension connector thereby raising and lowering the user within the framework when connected thereto.

Other aspects of the present invention will be apparent in view of the following description and claims.

### BRIEF DESCRIPTION OF THE DRAWINGS

FIG. 1 is a perspective of a first embodiment of a physical therapy support device of the present invention;

FIG. 2 is a perspective of a framework of the physical therapy support device of FIG. 1;

FIG. 3 is a front elevation of the framework of FIG. 2;

FIG. 4 is a right side elevation of the framework of FIG. 2;

FIG. 5 is a detail perspective illustrating a height adjuster mechanism;

FIG. 6 is a perspective similar to FIG. 2 but having a hand support and forearm supports;

FIG. 7 is a detail perspective illustrating the hand support;

FIG. 8 is a detail perspective illustrating the forearm supports;

FIG. 9 is a perspective similar to FIG. 2 but having an attached removable seat;

FIG. 10 is another perspective showing the removable seat;

FIG. 11 is a perspective of a first embodiment of a harness assembly;

FIG. 12 is another perspective of the harness assembly of FIG. 11;

FIG. 13 is a perspective of a second embodiment of a harness assembly;

FIG. 14 is a perspective of a third embodiment of a harness assembly;

FIG. 15 is a perspective of a physical therapy support device of a second embodiment of the present invention;

FIG. 16 is a perspective of a fourth embodiment of a harness assembly;

FIG. 17 is another perspective of the harness of FIG. 16;

FIG. 18 is a perspective of a user wearing the harness of FIG. 16;

FIG. 19 is another perspective of a user wearing the harness of FIG. 16;

FIG. 20 is a section of the harness of FIG. 16 taken in the plane of line 20-20 of FIG. 16;

FIG. 21 is another perspective of the harness of FIG. 16 with parts broken away to show hidden features;

FIG. 22 is a perspective of a back plate for use with the harness of FIG. 16;

FIG. 23 is another perspective of the back plate of FIG. 22;

FIG. 24 is a perspective of a fifth embodiment of a harness assembly worn by a user;

FIG. 25 is another perspective of the harness and user of FIG. 24;

FIG. 26 is a perspective of a sixth embodiment of a harness assembly;

FIG. 27 is a perspective of a second embodiment of a physical therapy support device of the present invention;

FIG. 28 is a fragmentary perspective of the physical therapy support device of FIG. 27 illustrating a hoist in which a portion of a housing is removed to reveal a coil spring;

FIG. 29 is a fragmentary section of the physical therapy support device of FIG. 27 taken in the plane of line 29-29 of FIG. 28 illustrating the hoist in a first or raised position;

FIG. 30 is a fragmentary section of the physical therapy support device similar to FIG. 29 but illustrating the hoist in a second or lower position; and

FIG. 31 is a top plan of the physical therapy support device of FIG. 27.

Corresponding reference characters indicate corresponding parts throughout the drawings.

### DETAILED DESCRIPTION OF THE DRAWINGS

Referring to FIG. 1, a physical therapy support device incorporating a first embodiment of the present invention is designated in its entirety by the reference number 20. The support device 20 generally includes a portable scaffold or framework, generally designated by 22, sized for surrounding a user or patient P and a harness assembly, generally designated by 24, for supporting a user while undergoing physical therapy. The harness assembly 24 suspends the user from the framework 22. Certain auxiliary accessories used in combination with the support device 20 are also shown in FIG. 1. These accessories include a hand support 30, forearm supports 32, and a seat 34.

As shown in FIGS. 2-4, the framework 22 includes a frame, generally designated by 40, connected to selectively extendable legs 42. The legs 42 extend downward from the frame 40 to casters 44 that permit the framework 22 to move freely about. The frame 40 is an assembly of an upper frame sub-assembly or member, generally designated by 46, and a mid frame sub-assembly or member, generally designated by 48. The upper frame member 46 includes four anchor brackets 50 to which the harness assembly 24 (FIG. 1) is attached. Although the framework 22 may have other numbers of legs without departing from the scope of the present invention, five legs 42 are connected to the mid frame member 48 by height adjuster mechanisms, generally designated by 52. Although the framework 22 may be made of other materials without departing from the scope of the present invention, in one embodiment the framework 22 is made of cylindrical aluminum tubing. Preferably, the materials used to make the framework 22 are sufficiently strong to support a user and sufficiently light to permit the user to easily move the framework. As will be appreciated by those skilled in the art, in the illustrated embodiment the tubing is cut into desired lengths, bent to shape, and welded together to form parts that are connected by screw fasteners to form the framework 22. Because the parts are connected by screw fasteners, the framework 22 may be disassembled for shipment and storage.

As shown in FIG. 1, the framework 22 includes an opening 54 at a rearward side, permitting a user to enter the framework. The user can either enter the framework 22 in a standing position by walking into the framework or the framework can be wheeled around the user while standing or sitting.

As shown in FIG. 5, the legs 42 are telescopically received in vertical tubing forming lower ends of the mid frame member 48 (FIG. 2). The tubing at the lower ends of the mid frame member 48 has a series of evenly spaced holes 60 extending upward along an outward facing side of the tubing. Although the holes 60 may be have other spacing without departing from the scope of the present invention, in one embodiment the holes are spaced at about one inch intervals. Each of the legs 42 includes a spring loaded detent 62 that engages a selected hole in the series of holes 60 to hold the leg in position. The legs 42 may be extended and retracted to raise and lower the framework 22, respectively, to change a height of the upper frame member 46 and thereby adjust the framework 22 to accommodate different sized users. As will be appreciated by those skilled in the art, the legs 42 are extended or retracted by pushing the detent 62 to disengage it from the corresponding hole 60 while moving the leg to align the detent with another hole.

FIGS. 6 and 7 depict the framework 22 having the hand support 30, which provides hand grips similar to those found on a conventional walker to permit the user to support at least a portion of the user's weight on the hand support or to move the framework 22 into a desired position and from station to station. As further illustrated in FIG. 7, the hand support 30 includes a mount 70 attached to mid frame member 48 and a handlebar 72 releasably attached to the mount. The mount 70 includes a rectangular section having a series of vertically spaced holes 74, each of which extend horizontally through the mount. The handlebar 72 has a clevis 76 sized to receive the rectangular section of the mount 70 so that opposing openings 78 in the clevis are aligned with one of the vertically spaced holes 74 in the mount. A pin 80 is inserted through the opposing openings 78 and corresponding hole 74 to hold the handlebar 72 in position. The series of holes 74 in the mount permit a height of the handlebar 72 to be adjusted.

FIGS. 6 and 8 depict the framework 22 having forearm supports 32 that the user can lean on for support. As further illustrated in FIG. 8, each forearm support 32 includes a mount 90 attached to the mid frame member 48 and an armrest 92 releasably attached to the mount. The mount 90 includes a rectangular section having a series of vertically spaced holes 94, each of which extend horizontally through the mount like the handlebar mount 70. The armrest 92 has a clevis 96 sized to receive the rectangular section of the mount 90 so that opposing openings 98 in the clevis are aligned with one of the vertically spaced holes 94 in the mount. A pin 100 is inserted through the opposing openings 98 and corresponding hole 94 to hold the armrest 92 in position. The series of holes 94 in the mount 90 permits heights of the armrests 92 to be adjusted. The armrest 92 has a contoured upper surface 102 for accommodating a user's forearm and a post 104 positioned for the user to grasp. The armrest 92 is attached to an articulating joint 106 that permits the armrest to be pivot about two perpendicular axes to adjust its position.

FIGS. 9 and 10 illustrate a removable seat 34 spanning the mid frame member 48 for the user to sit on such as when resting. The seat 34 includes a platform 110 slidably held on a flexible web 112 that is clamped to the mid frame member 48 at one end 114 and releasably fastened to the frame member at the other end by pins 116 inserted though two parallel series of holes 118 in the web for adjusting a height of the seat 34.

Returning to FIG. 1, the harness assembly 24 is releasably suspended from the four anchor brackets 50 of the upper frame member 46 for suspending the user in the framework 22. The harness assembly 24 may take one of several forms.

For example, as shown in FIGS. 11 and 12, the harness assembly 24 may include a truss or torso support 120 sized to encircle a user's torso. The torso support 120 is made of a flexible sheet 122 lined with foam 124. The support 120 includes straps 126 for fastening the support to the user. Webbing 130 is threaded through rings 132 on the torso support 120 to form leg loops 134 sized for receiving sand surrounding a leg of the user. The webbing 130 also extends upward from the rings 132 to form suspenders 136 that are connected to the framework 22.

FIG. 13 illustrates a second embodiment of the harness assembly, generally designated by 140 having a flexible torso support 142 made of a canvas material fastened around the user by belts 144 having hook and loop closures. As with the assembly 24 of the first embodiment, webbing 146 threaded through rings 148 on the support 142 form leg loops 150 and suspenders 152 that connect to the framework 22.

FIG. 14 illustrates a third embodiment of the harness assembly, generally designated by 160 having a flexible torso support 162 including a tensioned cord 164 wrapped around posts 166 on the support. The cord 164 is tightened around an adjustment reel 168 for adjusting the fit of the support on the user. Leg loops 170 extend downward from the support 162 and shoulder straps 172 extend upward from the support. The shoulder straps 172 have loops 174 for attaching the straps to separate suspenders (not shown) that are connected to the framework 22. In some embodiments, the suspenders have adjustable lengths.

FIG. 15 depicts an alternative embodiment of the physical therapy support device, generally designated by 180, in which the harness assembly 182 includes a belt 184 that is connected to a lower framework 186.

FIGS. 16-21 illustrate a fourth embodiment of the harness assembly of the present invention, generally designated 200. The harness assembly 200 includes a support 202 that is connected to a torso connector 204, leg loops 206, and suspenders 208. As shown best in FIGS. 20-21, the support 202 includes a back plate 210 having a shape corresponding to the shape of a user's back for supporting the user's back in a predetermined configuration. The torso connectors 204 are connected to the sides of the support 202 for engaging the user's torso and releasably connecting the back plate 210 to the user's torso adjacent the user's back. The leg loops 206 are connected to the bottom of the support 202. The loops 206 are sized for surrounding the user's leg to grasp the user around the leg fastening the support to the user's leg and support him. The suspenders 208 include shoulder straps 212 and suspension rings 214. The suspenders 208 may also include connectors (not shown) for connecting the suspension rings to a framework. Alternatively, the suspenders themselves may connect directly to the framework without intermediate connectors. The suspenders 208 extend outward from the support 202 for suspending the user, who is connected to the support (i.e., wearing the harness assembly), within the framework.

As shown in FIGS. 20 and 21, the support 202 includes a flexible housing 216 having a compartment or sleeve adapted to receive the back plate 210. In some embodiments, the flexible housing 216 is formed from a fabric material, optionally having a compartment opening (not shown) adapted to permit the insertion and removal of back plate 210 into and out of the flexible housing 216. Further, the compartment opening can be adapted to releasably secure the back plate 210 in the flexible housing 216. In one embodiment, the compartment opening is fitted with VELCRO® fasteners to secure an inserted back plate 210 in the housing 216. However, other releasable securement mechanisms may also be used without departing from the scope of the present invention.

In some embodiments, the back plate 210 (FIGS. 22 and 23) has a vertical length that extends in use along a user's spine from a lower portion 218 adjacent a user's waist upward to an upper portion 220 adjacent a user's scapula. More particularly, in the illustrated embodiment, the plate 210 extends from a lower edge adjacent the user's waist to an upper edge adjacent the user's scapula. In the illustrated embodiment, the back plate 210 has an S-shaped front surface contour to conform to the user's back. In some therapies, it may be desirable to mold or cast a user-specific back plate to conform to the shape of a particular user's back. In other therapies, it may be desirable to fit a user with a back plate that is shaped to support the user in a generic desirable posture. The back plate 210 may be selected from a group of interchangeable back plates.

In one embodiment, the back plate 210 is formed of a resiliently flexible material and has a substantially uniform thickness. Where therapeutically appropriate, the back plate 210 may also be substantially rigid without departing from the scope of this invention. Though other materials may be used, suitable back plate materials include plastics and fiber reinforced polymers. In embodiments in which the flexible housing 216 includes a compartment opening, the back plate 210 may be interchangeably selected from a group of back plates varying in stiffness. To prevent user discomfort, it may be desirable for the support 202 to include a layer of foam or other cushion material (not shown) between the back plate 210 and the flexible housing 216. Such a layer might absorb some of the concentrated pressures on a user's back, particularly around the edges of the back plate 210.

In the illustrated embodiment, the harness assembly 200 includes a torso connector 204 for engaging a user's torso. The torso connector 204 includes two waist straps 205. Each waist strap 205 comprises corresponding halves 222A, 222B attached to opposite sides of the support 202. In one embodiment, each waist strap half 222A, 222B is sewn to the support 202. The waist straps may be releasably secured to one another to releasably fasten the back plate 210 to the user's torso. In the illustrated embodiment, a side detent buckle 224 is used to releasably secure the corresponding strap halves 222A, 222B. However, other securement mechanisms may be used without departing from the scope of the present invention. For example, waist strap halves 222A, 222B may be joined by VELCRO® fasteners. In addition, securement mechanisms may be combined. Each strap half 222A, 222B includes webbing 226 and a foam-reinforced fabric member 228 that more directly engages the user's torso. The side detent buckle 224 includes at least one strap opening through which the webbing 226 may be looped to allow adjustment for accommodating users of differing girth.

In the illustrated embodiment, corresponding leg loops 206 and shoulder straps 212 are formed integrally from a single strap member 230. Each strap member 230 has a first or upper end 232 and a second or lower end 234. The first end 232 of each strap member 230 is sewn to the top of the support. In use, each strap member 230 extends downward from its first end 232 adjacent a user's shoulder and chest. The strap members 230 extend through openings between the webbing 226 and foam-reinforced fabric members 228 and through a first channel in a crossover strap guide 236. The first end of each strap member 232, along with the support 202 and the upper waist strap 205, define a shoulder opening 242. The second end of the strap member 234 forms a loop defining a leg opening 244, into which a user's leg may be inserted. The second end of the strap member 234 extends through the second channel in the crossover strap guide 236 and is sewn into the lower portion of the support 202. In some embodiments, the leg loops 206 include releasable securement mechanisms for releasably securing each loop to a user's leg. In the illustrated embodiment, a side detent buckle 238 is used, but other securement mechanisms may also be used. A shoulder strap connector 240 may be used to tether the shoulder straps 212 to one another providing a better fit across the user's chest.

To position a user in the harness assembly 200, each of the buckles 224, 238 should be unfastened. With the support 202 positioned adjacent the user's back, the user's arms are passed through shoulder openings 242, and the torso strap halves 222A, 222B are fastened together securing the torso connector 204 to the user's torso. Adjustments to the webbing members 226 may be made to ensure the torso connectors 204 firmly engage the user's torso. The second end of each strap member 234 should be pulled down through the first channel in the crossover strap guide 236 to pull each shoulder strap 212 against the user's chest and shoulder. The leg loops 206 are secured in place to separately surround and grasp each of the user's legs. The suspension rings 214 may then be secured to suspender connectors in a physical therapy support device such as the ones illustrated in FIGS. 1-10 and FIGS. 27-31 to suspend a user in a framework.

FIGS. 24 and 25 depict an alternative embodiment of a physical therapy support device 250. The harness assembly 250 includes a support 252 and a torso connector 254, and two shoulder straps 260. As in the embodiment illustrated in FIGS. 16-23, the support 252 includes a back plate 258, which may include any of the features described in reference to back plate 210. The torso connector 254 includes two waist straps 255 and may be connected to the support 252 to releasably connect the support to the user's torso adjacent the user's back. The primary differences between the embodiment of FIGS. 24 and 25 and that of FIGS. 16-23 is the absence of leg loops and suspension rings. Each of the shoulder straps 260 includes a first and second end. The first end of each shoulder strap 260 is sewn to the top of the support 252. The second end of each shoulder strap is sewn to the lower waist strap 255. Shoulder openings 264 are defined by the shoulder straps 260, the support 252, and the upper waist strap 255. The harness assembly 250 need not be used with a physical therapy support framework for suspending the user. It may be used as an independent back brace or support in applications such as weight lifting, truck driving, etc. In one embodiment, the bottom of the support 252 includes a releasably attachable opening (not shown). The opening may provide access to a pouch suitable for retaining lumbar support foam in the support 252. Additionally, the pouch may be suitable for retaining an ice pack, heating pad, or other therapeutic aid.

FIG. 26 illustrates another alternative embodiment of a harness assembly 300. The harness assembly 300 includes many elements that are similar to the elements of the harness assembly 200. (Similar elements are given the same reference number, plus one-hundred.) The harness assembly 300 includes a support 302, a torso connector 304, two leg loops 306, and two suspenders 308. Like harness assembly 200, the support 302 includes a back plate (not shown) having a shape corresponding to a user's back for supporting the user's back in a predetermined configuration. The back plate of the harness assembly 300 may include the features discussed in reference to the back plate 210. The single torso connector 304 acts as a waist strap for releasably surrounding the waist of the user. The torso connector 304 engages the user's torso and releasably connects the back plate to the user's torso adjacent the user's back. The suspenders 308 include shoulder straps 312 and suspension rings 314. The support 302 may include a pouch (not shown) positioned near the bottom of the support for receiving lumbar support foam, an ice pack, heating pad, or other therapeutic aid.

The harness 300 has shoulder straps 312 that are adjustable independently from the leg loops 306. Generally, the shoulder straps 314 are connected to the torso connector or waist strap 304 and loop above the shoulder of the user. The shoulder straps 314 include foam-reinforced fabric members 348 and adjustment straps 350. The foam-reinforced fabric members 348 are attached to the top end of the support 302, and the adjustment straps 350 are attached to the torso connector 304. In the illustrated embodiment, each adjustment strap 350 is adjustably secured to a foam-reinforced fabric member 348 by a loop fastener 351, though other fasteners may also be used without departing from the scope of the present invention. The shoulder straps 312 further include a plurality of chest tether connectors 352 for selectably connecting the chest tether 340 to one of a plurality of vertical positions along the shoulder straps.

The leg loops 306 include foam-reinforced fabric members 354, leg loop straps 356, and a hip restraint strap 358. The fabric members 354 are each attached to the support 302. In the illustrated embodiment, the fabric members 354 are each sewn onto the bottom of the support 302. The fabric members 354 each include a hip bolster 360 and a leg pad 362. Each leg loop strap 356 extends from a leg loop pad 362 and connects to a fabric member 354 at a position on or adjacent the hip bolster 360. Thus, a length of each leg loop strap 356, in combination with a fabric member 354, forms a loop for surrounding, supporting, and fastening to the user's leg. Preferably, each leg loop strap 356 includes an adjustable connector 364. In the illustrated embodiment, each adjustable connector 364 comprises a side detent buckle having an opening to receive the strap for adjusting the length of the leg loop strap 356 that forms a portion of the leg loop 306. Each of the hip bolsters 360 is releasably and adjustably connected together with the hip restraint strap 358. In the illustrated embodiment, the hip restraint strap 358 includes opposite side detent buckles 366, each having an opening for adjusting the length of strap between each of the hip bolsters 360. In use, the hip restraint strap 358, in combination with each of the fabric members 354, surround the hips of the user to provide stability and position the user's hips with respect to the user's back.

In use, the user extends each of his arms through one of the shoulder straps 312. The torso connector 304 is connected around the torso of the user. In the illustrated embodiment, VELCRO® fasteners (not shown) may be used to initially attach the fabric members 328 to one another. The buckle 324 may then be used to connect the webbing 326 of each strap 322A, 322B to one another. Adjustment straps 350 may be used to change the length of each shoulder strap 312, so that each shoulder strap comfortably engages the user's shoulder and chest. The leg loops 306 are fastened at the connectors 364 and adjusted to surround the user's leg. The hip restraint strap 358 may then be connected to the fabric members 354 and be adjusted to support the hips of the user. The suspension rings 314 may be secured to suspender connectors in a physical therapy support device such as those illustrated in FIGS. 1-10 and FIGS. 27-31 to suspend a user within a framework.

FIGS. 27-31 illustrate an alternative embodiment of a physical therapy support device which is designated in its entirety by the reference number 420. Many features of the physical therapy support device 420 are common with the physical therapy support device illustrated in FIGS. 1-10. (Similar features are designated as in the previous embodiment, plus four-hundred.) The physical therapy support device includes a portable scaffold or framework 422, much like the framework 22 of FIGS. 1-10, which is sized for at least partially surrounding a user. The framework includes a frame member 440 having forward and rearward ends and a plurality of legs 442 extending downward from the frame member to casters 444 positioned at lower ends of the legs. The frame member 444 is somewhat different from the frame member 44. Particularly, the front vertically oriented frame columns 520 have bends so they include angled sections 522 that allow a user to ergonomically grasp the frame columns 520 for support and balance without bending his or her wrists abnormally.

Of the auxiliary accessories discussed with respect to the physical therapy support device 20, only a hand support 430 is shown fitted to the physical therapy support device 420. However, it should be understood that any auxiliary accessories discussed with respect to the physical therapy support device 20 may also be fitted to the physical therapy support device 420 without departing from the scope of the invention. The hand support 430 provides two pairs of hand grips or handlebars 472A, 472B similar to those found on a conventional walker. The first pair of handlebars 472A extends into the framework 422 toward the user. The handlebars 472A permit the user to support at least a portion of the user's weight on the hand support 430 or to move the framework into a desired position. The second pair of handlebars 472B extends out and away from the framework 422 and the user. The handlebars 472B provide a gripping location for a therapist or other assistant when moving the physical therapy support device 428 to a desired position. The assistant can grasp the handlebar 472B with one or both hands to steady or direct the movement of the physical therapy support device 420.

The physical therapy support device 420 further includes a hoist 530 for adjustably suspending the user in the framework. The hoist 530 includes suspension connectors 532 that are releasably connectable to the harness. Suspension cables 534 support the suspension connectors 532. As will be described in more detail below, the suspension cables 534 are adapted to selectively raise and lower the suspension connectors 532 and in turn raise and lower the user within the framework 522. A winch 536 is mounted on the frame member 440 and connected to the suspension cables 534. The winch 536 winds and unwinds the cables 534 to raise and lower the suspension connectors 532.

In the illustrated embodiment, the winch 536 includes a guide cable 544 wound around a spool 546. As the winch 536 is wound or unwound, the spool 546 rotates and the guide cable 544 is taken in or let out. The guide cable 544 is operatively connected to the suspension cables 534 by a yoke 548. The yoke 548 is attached to a first end of each of the suspension cables 534. In the illustrated embodiment, the yoke 548 holds the first end of each of the suspension cables 534 laterally spaced from the other at a distance approximating a shoulder width of the user. In the illustrated embodiment, the yoke 548 has a length that approximates the shoulder width of a user, and the first end of each of the suspension cables 534 is attached adjacent an opposite end of the yoke. The guide cable 544 is attached to the center of the yoke 548.

As shown in FIGS. 28-31, a track system 538 is mounted on the frame member 440. The track system 538 includes a track 540 that is mounted on the frame 440 and extends between the front and back of the frame. The track system 538 further includes a guide 542 for holding the suspension cables 534. The guide 542 is configured to travel along the track 540 to adjust the position of the user relative to the framework 422. More particularly, as the guide 542 moves along the track 540, the user is raised and lowered relative to the framework 522. In one embodiment, the guide 542 includes a car having wheels configured to roll along rails of the track 540. The wheels are rotatably secured to a guide-car frame attached to a yoke securement means for securing the guide 542 to the yoke 548. Alternative guides 542 may also be used without departing from the scope of the invention. Preferably, the guide 542 is biased toward the forward end of the track 540. As shown in FIG. 28, the guide 542 is biased toward the forward end of the track by a coil spring 550 affixed to the forward end of the track 540. In the illustrated embodiment, the spring 550 includes a guide end (not shown) that is connected to the guide 542. The spring 550 applies a constant tension against the guide 542 to bias the guide toward the forward end of the physical therapy support device 520. Other biasing mechanisms may also be used within the scope of this invention.

The winch 536 is mounted on the frame member 440 and is operatively connected to the guide 542 for moving the guide along the track 540. The yoke 548 is connected to a lower end of the guide 542, and the guide cable 544 extends forward from the winch 536 to the yoke. As the guide cable 544 is wound or unwound from the spool 546, the yoke 548 moves parallel to the track 540, causing the laterally spaced suspension cables 534 to also move parallel with the track 540 and in line with the guide cable 544. Because the guide 542 is biased toward the forward end of the track 540 by the spring 550, the guide cable 544 is held in tension so the position of the guide is determined by the length of guide cable let out between the spool 546 and the yoke 548.

Each of the suspension cables 534 extends forward from the yoke 548 to a cable guide 552 mounted on the forward end of the frame member 440. In the illustrated embodiment, the cable guide 552 includes a sheave sized for engaging the suspension cable 534. The sheave is rotatably mounted on a mounting plate fixed to the forward end of the frame member 440. In one embodiment, the cable guides 552 are fixed to the frame member 440 at a spacing that is approximately equal to the lateral spacing of the first ends of the suspension cables 534. From each cable guide 552, each suspension cable 534 extends rearward to a load cell 554 at the rearward end of the frame member 440. A harness guide 556, which is rotatably attached to a suspension connector 532, is mounted on each suspension cable 534 between the cable guide 552 and the mount 554. The harness guide 556 includes a pulley having a perimeter channel sized for engaging the suspension cable 534. In the illustrated embodiment, the suspension connector 532 is mounted beneath the harness guide 556 and the suspension cable 534 so the suspension cable extends between the harness guide and the suspension connector.

A battery pack 558 supplies power to the winch 536, and a control unit 560 controls operation of the winch. Using the control unit 560, a user may control the winch 536 to wind or unwind the cable onto or from the spool 546. Limit switches 562, 564 shut off power to the winch 536 if the guide 542 nears the end of the track 540. In FIG. 29, the hoist 530 is shown in a first position in which the guide cable 544 is terminally wound around spool 546. In this position, the guide 542 contacts the rear limit switch 562, preventing the winch from raising the harness guides 556 farther. At the opposite extreme (not shown), the guide 542 contacts the forward limit switch 564, preventing the winch from lowering the harness guides 556 farther.

FIGS. 29 and 30 show the basic operation of the hoist system 530. In FIG. 29, the hoist 530 is shown in the fully wound position. A user may provide an input to the control unit 560 to unwind the guide cable 544. As the guide cable 544 is unwound from spool 546, the guide 542 moves along the track 540 from the rearward end to the forward end under the influence of the spring 550. The yoke 548 moves parallel to the guide 542, and the suspension cables 534 move parallel to the yoke. The suspension cables 534 are biased forward and downward by the weight of the harness guides 556, suspension connectors 532, and the harness and user. Thus, as the yoke 548 moves forward, the harness guides 556 and suspension connectors 532 are lowered. The guide cable 544 may be unwound until the guide 542 reaches the forward limit switch 564. To raise the harness guides 556 and suspension connectors 532, the guide cable 544 is wound around the spool 546. The guide 542 moves rearward along the track 540, moving the yoke 548 and the suspension cables 534 along with it. As the yoke 548 moves rearward, the harness guides 556 and the suspension connectors 532 are raised.

A harness assembly (not shown), which may be suitably chosen from the group of harness assemblies including harness assemblies 200, 250, and 300, may be suspended from the harness guides 556 for holding a user. In operation, any of the harness assemblies 200, 250, or 300 may be connected to the suspension connectors 532 at their suspension rings 214, 262, or 314. Thus, as the guide cable 544 is unwound from the spool 546, the suspended harness assembly and the user are lowered. As the guide cable 544 is wound around the spool 546, the suspended harness assembly and the user are raised. Using the control unit 560 to control the winch 536, a user can be raised or lowered within the framework 422 to a desired level corresponding to a predetermined amount of support. The load cells 554 can send information to the control unit 560, allowing the assistant to know how much of the user's weight is being supported by the framework.

Having described the invention in detail, it will be apparent that modifications and variations are possible without departing from the scope of the invention defined in the appended claims.

When introducing elements of the present invention or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A harness assembly for suspending a user, said harness assembly comprising:
a support including a back plate having a shape corresponding to a user's back for supporting the user's back in a predetermined configuration,
a torso connector connected to the support for engaging the user's torso and releasably fastening the support to the user's torso adjacent the user's back;
a leg loop connected to the support and sized for surrounding the user's leg to fasten the support to the user's leg; and
a suspender extendable outward from the support for suspending the user fastened to the support.

2. The harness assembly of claim 1, wherein the back plate has a vertical length extending in use along a user's spine from a lower portion adjacent a user's waist upward to an upper position adjacent a user's scapula.

3. The harness assembly of claim 1, wherein the back plate has an S-shaped front surface contour to conform to the user's back.

4. The harness assembly of claim 1, wherein the back plate comprises a resiliently flexible material.

5. The harness assembly of claim 1, wherein the back plate comprises a material having a substantially uniform thickness.

6. The harness assembly of claim 1, wherein the torso connector comprises at least one waist strap for surrounding the waist of the user.

7. The harness assembly of claim 6, wherein the suspender comprises a shoulder strap connected to the waist strap and looped above a shoulder of the user.

8. The harness assembly of claim 7, wherein the shoulder strap extends from the leg loop.

9. The harness assembly of claim 7, wherein the shoulder strap comprises a suspension ring configured for releasably attaching to a suspender.

10. The harness assembly of claim 1, wherein the back plate is selected from a group of interchangeable back plates varying in stiffness.

11. A harness assembly for supporting a back of a user comprising:
a support including a back plate having a shape corresponding to a user's back for supporting the user's back in a predetermined configuration, the back plate having a vertical length extending in use along a user's spine from a lower portion adjacent a user's waist upward to an upper position adjacent a user's scapula;
a torso connector connected to the support for engaging the user's torso and releasably fastening the support to the user's torso adjacent the user's back; and
a shoulder strap connected to the torso connector and looped above a shoulder of the user.

12. A physical therapy support device comprising:
a framework sized for at least partially surrounding a user, the framework having a frame member and a plurality of legs extending downward from the frame member to casters positioned at lower ends of the legs; and
a hoist secured to the frame member for adjustably suspending the user within the framework, the hoist comprising a suspension connector releasably connectable to the user and a suspension cable connected to the connector and adapted to selectively raise and lower the suspension connector thereby raising and lowering the user within the framework when connected thereto.

13. The physical therapy support device of claim 12, wherein the hoist further comprises a winch mounted on the frame member and connected to the suspension cable for raising and lowering the suspension cable to raise and lower the suspension connector.

14. The physical therapy support device of claim 12, wherein the hoist further comprises a track mounted on the frame member extending between a forward end and a rearward end, and a guide for holding the suspension cable, the guide configured to travel along the track to adjust a position of the user relative to the framework.

15. The physical therapy support device of claim 14, wherein the user is raised and lowered relative to the framework as the guide travels along the track.

16. The physical therapy support device of claim 14, wherein the guide is biased toward the forward end of the track.

17. The physical therapy support device of claim 14, wherein the hoist further comprises a winch mounted on the frame member, the winch including a spool operatively connected to the guide for moving the guide along the track.

18. The physical therapy support device of claim 17, further comprising a yoke mounted on the guide and a guide cable attached to the yoke, said guide cable being wound on the spool so that the yoke moves parallel to the track as the winch winds and unwinds the guide cable.

19. The physical therapy support device of claim 18, wherein:
the suspension cable extends from the yoke forward to a cable guide mounted on the frame member and from the cable guide rearward to a mount mounted on the frame member; and
the support device further comprises a harness guide mounted on the suspension cable between the cable guide and the mount, and a harness assembly suspended from the harness guide for holding a user, said assembly being raised and lowered as the yoke moves parallel to the track.

20. The physical therapy support device of claim 19, wherein the harness assembly comprises:
a back plate having a shape corresponding to a user's back for supporting the user's back in a predetermined configuration,
a torso connector for releasably fastening the support to the user's torso adjacent the user's back; and
a user suspension system operatively connected to the back plate for suspending harness assembly from the harness guide.
